(19) United States
(12) Patent Application Publication
(10) Pub. No.: US 2004/0044393 A1
(43) Pub. Date: Mar. 4, 2004

(54) Implant system

(75) Inventors: Orit Yarden, Glvat Shmuel (IL); Vitaly Fastovsky, Haifa (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(73) Assignee: Remon Medical Technologies Ltd.

(21) Appl. No.: 10/227,832

(22) Filed: Aug. 27, 2002

(51) Int. Cl. A61F 2/06

(52) U.S. Cl. 623/1.2

(57) ABSTRACT

A self-expansible structure for implantation in a body cavity is provided. The structure has a generally tubular outline, a nominal length, a nominal diameter, and a unique behavior under constraint. The unique behavior is expressed by a transition diameter, at which a constrained behavior of the structure changes so that, at a constraining diameter smaller than the transition diameter, the structure conforms to the constraint by decreasing the structure's diameter, to below the nominal diameter, and elongating beyond the nominal length, while at a constraining diameter larger than the transition diameter but smaller than the nominal diameter, the structure conforms to the constraint by decreasing the structure's diameter below the nominal diameter, while substantially maintaining the nominal length. Thus, the structure is operable for insertion into a body cavity, via a catheter, having a catheter inner diameter smaller than the transition diameter, by decreasing the structure's diameter, and elongating, and the structure is operable for implantation in the body cavity, having a cavity inner diameter greater than the transition diameter but smaller than the nominal diameter, by decreasing the structure's nominal diameter, while substantially maintaining the nominal length. The value of the transition diameter may be varied and controlled by the manner of constraining in the catheter, during insertion.

In the preferred embodiment, the structure is formed of two closed wire constructions, shaped as symmetric wings, and has no exposed tips. The structure may be a stent. Alternatively, the structure may be an anchor, for mounting an incorporeal device thereon.

### FIELD OF THE INVENTION

The present invention relates generally to implantations of intracorporeal devices in body cavities, and in particular, to a system and method of their anchoring in the body cavities.

### BACKGROUND OF THE INVENTION

Intracorporcal devices, implanted in body cavities for performing physiologically related tasks, have been described in the art. U.S. Pat. No. 6,277,078, to Porat et al., whose disclosure is incorporated herein by reference, describes an intrabody implantable biosensor for long-term, real-time monitoring of at least one parameter associated with heart performance. Additionally, U.S. patent application Ser. No. 09/872,129 (Publication No. 20010026111), to Doron et al., whose disclosure is incorporated herein by reference, describes an acoustic biosensor for monitoring physiological conditions at a particular implantation site within the body. Implantable biosensors have also been described in U.S. Pat. No. 5,368,040, to Carney, and U.S. Pat. No. 5,704,352, to Tremblay, whose disclosures are incorporated herein by reference.

Implantable biosensors are of particular importance in the diagnosis and management of heart disease. Unlike invasive procedures, which require a separate procedure each time information is sought, implantable biosensors provide long-term, real-time monitoring of physiological parameters after a single invasive procedure.

Intracorporeal biosensors and devices may be arranged on anchors, such as various stud and screw configurations, which pierce the tissue, as taught by U.S. Pat. No. 6,277,078, hereinabove. However, these configurations are disadvantageous, as they necessarily cause tissue injury and are difficult to retrieve once implanted.

EP Patent Application 0 928 598 A2, to Richter, et al., describes an implant system and a method for anchoring a sensor in a body cavity, wherein the sensor is coupled to an anchor. The anchor can be one or more dedicated anchoring rings, having a contracted condition, adapted for insertion into a cavity, and an expanded condition, adapted for anchoring by exerting pressure against the walls of the cavity.

In the expanded condition, the anchoring ring has a predetermined diameter. However, the actual size of the cavity in which anchoring takes place is not known precisely. If the diameter of the ring is too small in relation to the cavity size, then the biasing forces exerted by the ring against the walls of the cavity may be too low for anchoring. On the other hand, if the diameter of the ring is too large in relation to the cavity size, the biasing force exerted by the ring may be unnecessarily excessive and possibly damaging. Alternatively, a ring that is too large in relation to the cavity may assume a slanted position with respect to a length axis of the cavity, leading to insufficient biasing force.

Another disadvantage of EP Patent Application 0 928 598 A2, hereinabove, is that the length of the sensor essentially determines the length of the overall implant system. If the sensor is small, the implant system may be too short, in relation to its diameter, thus anchoring may be unstable.

U.S. patent application Ser. No. 2002/0077556A1, to Schwarts, describes another anchoring mechanism for implantable telemetric medical sensors.

Alternatively, as taught by U.S. Pat. No. 6,277,078, intracorporeal biosensors may be arranged on stents.

The Handbook of Coronary Stents (Rotterdam Thoraxcenter Group, Edited by P. W. Serruys and M. J. B. Kutryk, 2nd edition, 1998, Chapter 25, by Rafael Beyar, pp. 243-249) describes a stent known as the Coronary Cardiocoil, a self-expanding Nitinol coil stent that was developed by Mederonic InStent. This stent is formed as a coiled wire and assures good adherence to the blood vessel wall even when expansion docs not reach the nominal stent diameter.

Since the self-expanding coil adjusts to the cavity size, it gives answer to situations where the cavity size is not known precisely. However, the length of the self-expanding coil varies with the extent of expansion. When expansion does not reach the nominal diameter, the length is greater than the nominal length. This may cause problems in situations where space is tight, and it is desired to know the precise length of the deployed stent.

U.S. Pat. No. 6,273,908, to Ndondo-Lay, whose disclosure is incorporated herein by reference, describes a ribcage-like stent, which may adjust to a cavity size while maintaining the nominal length. This stent consists of a flexible serpentine backbone with a plurality of outward-projecting appendages on both the right and left sides, forming a substantially circular cross-section. The appendages are shifted to respect with each other so as to intertwine. The stent is adapted for keeping a blood vessel open with a minimum degree of recoil and shortening lengthwise. Since the appendages do not interconnect they permit the stent to be compressed or expanded over a wide range of diameters, while still maintaining the significant mechanical force required to prevent a vessel from recoiling or collapsing.

Both the Nitinol coil stent of Mederonic InStent and the ribcage-like stent of U.S. Pat. No. 6,273,908 have exposed tips that may injure or scratch the tissue. Furthermore, both are deployed asymmetrically—the appendages of U.S. Pat. No. 6,273,908 are shifted with respect with each other, so as to intertwine, and coil are inherently asymmetric. In consequence, bending moments may be generated and may lead to toppling. Moreover, stents are mechanically designed to provide structural support to the vessel in which they are deployed. As such, they can be unnecessarily bulky, and may cause stenosis when deployed in a healthy vessel portion.

There is thus a need for anchors for implanting intracorporeal devices, devoid of the above limitations.

### SUMMARY OF THE INVENTION

According to an aspect of the present invention, there is provided a self-expansible structure, for implantation in a body cavity, the structure comprising:

a generally tubular outline;

a nominal length;

a nominal diameter; and

a transition diameter, at which a constrained behavior of the structure changes so that:

at a constraining diameter smaller than the transition diameter, the structure conforms to the constraint by decreasing the structure's diameter, to below the nominal diameter, and elongating beyond the nominal length, and

at a constraining diameter larger than the transition diameter but smaller than the nominal diameter, the structure conforms to the constraint by decreasing the structure's diameter below the nominal diameter, while substantially maintaining the nominal length,

wherein:

the structure is operable for insertion into a body cavity, via a catheter, having a catheter inner diameter smaller than the transition diameter, by decreasing the structure's diameter, while elongating, and

the structure is operable for implantation in the body cavity, having a cavity inner diameter greater than the transition diameter but smaller than the nominal diameter, by decreasing the structure's nominal diameter, while substantially maintaining the nominal length.

According to an additional aspect of the present invention, the structure is a stent, designed to provide structural support to a blood vessel.

According to an alternative aspect of the present invention, the structure is an anchor, designed to support an intracorporeal device thereon.

According to an additional aspect of the present invention, the anchor further includes an intracorporeal device, mounted thereon, adapted for performing a physiologically related task at the body cavity.

According to yet an additional aspect of the present invention, the intracorporeal device is selected from the group consisting of a pressure sensor, a flow rate sensor, a temperature sensor, an oxygen concentration sensor, an ion concentration sensor, an impedance sensor, a sensor adapted for cardiac output assessment, a blood filter, a septal occluder, a coil, a detachable coil for aneurysm treatment, a graft, and a deflector.

According to an alternative aspect of the present invention, the anchor further includes a carrier for mounting an intracorporeal device thereon.

According to an additional aspect of the present invention, the nominal length is at least 25% greater than the nominal diameter.

According to an alternative aspect of the present invention, the nominal diagonal is at least 25% greater than the nominal diameter.

According to an additional aspect of the present invention, in the deployed state, the perimeter of the structure forms an arc that is at least as great as half the cavity perimeter.

According to yet an additional aspect of the present invention, in the deployed state, the perimeter of the structure forms an arc that is at least as great as two thirds the cavity perimeter.

According to still an additional aspect of the present invention, the structure is formed of a wire of a diameter of between 0.03 and 1.0 mm.

According to yet an additional aspect of the present invention, the structure is formed of a wire of a diameter of between 0.2 and 0.3 mm.

According to still an additional aspect of the present invention, the value of the transition diameter may be varied and controlled by the manner of constraining in the catheter, during insertion.

According to yet an additional aspect of the present invention, the structure is formed of at least one closed wire construction, thus having no exposed tips.

According to still an additional aspect of the present invention, the structure is formed of at least two closed wire constructions, thus having no exposed tips.

According to yet an additional aspect of the present invention, the structure defines a length axis, parallel to its length, and a point of midlength, on the length axis, and wherein the structure is symmetric with respect to a plane, orthogonal to the length axis, and crossing the length axis at the point of midlength.

According to still an additional aspect of the present invention, the structure is formed of at least one closed wire construction, thus having no exposed tips, and the structure defines a length axis, parallel to its length, and a point of midlength, on the length axis, and wherein the structure is symmetric with respect to a plane, orthogonal to the length axis, and crossing the length axis at the point of midlength.

According to yet an additional aspect of the present invention, the structure is formed of a shape memory alloy, having a martensite phase at temperatures below body temperature and an austenite phase at temperatures at and above body temperature, and wherein the structure is at the martensite phase, at the martensite temperatures, when constrained in the catheter, for insertion into the body.

According to still an additional aspect of the present invention, the structure is at the austenitic phase, at the austenitic temperatures, when implanted in the body cavity.

According to an alternative aspect of the present invention, the structure is at a stress-induced martensite phase, at the austenitic temperatures, when implanted in the body cavity.

According to still an alternative aspect of the present invention, the structure is formed of a shape memory alloy, having an austenite phase and a stress induced martensite phase at and above room temperature, and wherein the structure is at the stress-induced martensite phase, both when constrained in the catheter, for insertion into the body, and when implanted in the body cavity.

According to still an additional aspect of the present invention, the structure further includes at least-one inner component for engaging with a retrieval instrument.

According to another aspect of the present invention, there is provided an implant system, comprising:

a self-expansible structure, for implantation in a body cavity, the structure comprising:

a generally tubular outline;

a nominal length;

a nominal diameter; and

a transition diameter, at which a constrained behavior of the structure changes so that:

at a constraining diameter smaller than the transition diameter, the structure conforms to the constraint by decreasing the structure's diameter, to below the nominal diameter, and elongating beyond the nominal length, and

at a constraining diameter larger than the transition diameter but smaller than the nominal diameter, the structure conforms to the constraint by decreasing the structure's diameter below the nominal diameter, while substantially maintaining the nominal length,

wherein:

the structure is operable for insertion into a body cavity, via a catheter, having a catheter inner diameter smaller than the transition diameter, by decreasing the structure's diameter, while elongating, and

the structure is operable for implantation in the body cavity, having a cavity inner diameter greater than the transition diameter but smaller than the nominal diameter, by decreasing the structure's nominal diameter, while substantially maintaining the nominal length; and

an intracorporeal device, mounted on the self-expansible structure and adapted for performing a physiologically related task at the body cavity.

According to an additional aspect of the present invention, the intracorporeal device comprises at least two-intracorporeal devices.

According to an additional aspect of the present invention, the intracorporeal device comprises a power source.

According to yet an additional aspect of the present invention, the intracorporeal device comprises an extracorporeally energizeable power source.

According to still an additional aspect of the present invention, the intracorporeal device is capable of telemetric communication with an extracorporeal device.

According to another aspect of the present invention, there is provided a retrieval system, comprising:

a self-expansible structure, for implantation in a body cavity, the structure comprising:

a generally tubular outline;

a nominal length;

a nominal diameter; and

a transition diameter, at which a constrained behavior of the structure changes so that:

at a constraining diameter smaller than the transition diameter, the structure conforms to the constraint by decreasing the structure's diameter below the nominal diameter and elongating beyond the nominal length, and

at a constraining diameter larger than the transition diameter but smaller than the nominal diameter, the structure conforms to the constraint by decreasing the structure's diameter below the nominal diameter, while substantially maintaining the nominal length,

wherein:

the structure is operable for insertion into a body cavity, via a catheter, having a catheter inner diameter smaller than the transition diameter, by decreasing the structure's diameter, while elongating, and

the structure is operable for implantation in the body cavity, having a cavity inner diameter greater than the transition diameter but smaller than the nominal diameter, by decreasing the structure's nominal diameter, while substantially maintaining the nominal length,

wherein:

the structure includes at least one inner component for engaging with a retrieval instrument, and

the retrieval system includes a retrieval instrument, mounted on a retrieval catheter, adapted for percutaneous insertion into the body cavity, for engaging with the inner component and for retrieving the structure.

According to an additional aspect of the present invention, the structure further includes an intracorporeal device, mounted thereon, adapted for performing a physiologically related task at the body cavity.

According to another aspect of the present invention, there is provided a method of implanting a self-expansible structure, comprising:

providing self-expansible structure for implantation in a body cavity, the structure comprising:

a generally tubular outline;

a nominal length;

a nominal diameter; and

a transition diameter, at which a constrained behavior of the structure changes so that:

at a constraining diameter smaller than the transition diameter, the structure conforms to the constraint by decreasing the structure's diameter and elongating, and

at a constraining diameter larger than the transition diameter but smaller than the nominal diameter, the structure conforms to the constraint by decreasing the structure's diameter, while substantially maintaining the nominal length;

constraining the structure within a catheter, having a catheter diameter smaller than the transition diameter, wherein the structure conforms to the catheter by decreasing the structure's diameter and elongating;

inserting the catheter and structure to a body cavity, having a cavity diameter larger than the transition diameter but smaller than the nominal diameter; and

deploying the structure in the body cavity, wherein the structure conforms to the cavity by decreasing the structure's diameter, while substantially maintaining the nominal length.

According to still another aspect of the present invention, there is provided a self-expansible structure, for implantation in a body cavity, wherein the self-expansible structure is formed of at least one closed wire construction, thus having no exposed tips.

According to yet another aspect of the present invention, there is provided a self-expansible structure, for implantation in a body cavity, wherein the self-expansible structure is symmetric with respect to a plane, orthogonal to a length axis of the structure and crossing the length axis at a point of midlength of the structure.

According to still another aspect of the present invention, there is provided a self-expansible structure, for implantation in a body cavity, wherein;

the self-expansible structure is formed of at least one closed wire construction, thus having no exposed tips; and

the self-expansible structure is symmetric with respect to a plane, orthogonal to a length axis of the structure and crossing the length axis at a point of midlength of the structure.

According to yet another aspect of the present invention, there is provided a implant system comprising:

a self-expansible structure, for implantation in a body cavity, wherein the self-expansible structure is formed of at least one closed wire construction, thus having no exposed tips; and

an intracorporeal device, mounted on the self-expansible structure and adapted for performing a physiologically related task at the body cavity.

According to still another aspect of the present invention, there is provided a implant system comprising:

a self-expansible structure, for implantation in a body cavity, wherein the self-expansible structure is symmetric with respect to a plane, orthogonal to a length axis of the structure and crossing the length axis at a point of midlength of the structure; and

an intracorporeal device, mounted on the self-expansible structure and adapted for performing a physiologically related task at the body cavity.

According to yet another aspect of the present invention, there is provided a implant system comprising:

a self-expansible structure, for implantation in a body cavity, wherein;

the self-expansible structure is formed of at least one closed wire construction, thus having no exposed tips; and

the self-expansible structure is symmetric with respect to a plane, orthogonal to a length axis of the structure and crossing the length axis at a point of midlength of the structure; and

an intracorporeal device, mounted on the self-expansible structure and adapted for performing a physiologically related task at the body cavity.

The present invention successfully addresses the shortcomings of presently known configurations by providing a self-expansible structure for implantation in a body cavity. The structure has a generally tubular outline, a nominal length, a nominal diameter, and a unique behavior under constraint. The unique behavior is expressed by a transition diameter, at which a constrained behavior of the structure changes so that, at a constraining diameter smaller than the transition diameter, the structure conforms to the constraint by decreasing the structure's diameter, to below the nominal diameter, and elongating beyond the nominal length, while at a constraining diameter larger than the transition diameter but smaller than the nominal diameter, the structure conforms to the constraint by decreasing the structure's diameter below the nominal diameter, while substantially maintaining the nominal length. Thus, the structure is operable for insertion into a body cavity, via a catheter having a catheter inner diameter smaller than the transition diameter, by decreasing the structure's diameter, and elongating, and the structure is operable for implantation in the body cavity, having a cavity inner diameter greater than the transition diameter but smaller than the nominal diameter, by decreasing the structure's nominal diameter, while substantially maintaining the nominal length. The value of the transition diameter may be varied and controlled by the manner of constraining in the catheter, during insertion.

In the preferred embodiment, the structure is formed of two closed wire constructions, shaped as symmetric wings, and has no exposed tips. The structure may be a stent. Alternatively, the structure may be an anchor, for mounting an incorporeal device thereon.

### BRIEF DESCRIPTION OF THE DRAWINGS

The invention herein is described by way of example only, with reference to the accompanying drawings. The details presented in these drawings are merely examples provided to illustrate the preferred embodiments of the invention, and as such they represent what is believed to be the most useful and readily understood description of the principles and conceptual aspects of this invention. In this regard, no attempt is made to show structural details of the invention in more detail than is necessary for a fundamental understanding of the invention. These drawings, taken in conjunction with the description of the preferred embodiments, should make apparent to those skilled in the art how the several forms of the invention may be embodied in practice. In the drawings:

FIGS. 1A-1D: schematic illustrations of relationships between length and diameter of prior-art, self-expansible stents;

FIGS. 2A-2F: schematic illustrations of a relationship between length and diameter of a self-expansible structure, in accordance with a preferred embodiment of the present invention;

FIGS. 3A-3H: schematic illustrations of an implant system, in accordance with a preferred embodiment of the present invention;

FIGS. 4A-4D: schematic illustrations of implant systems, in accordance with other embodiments of the present invention;

FIG. 5: schematic illustration of an implant system, in accordance with yet another embodiment of the present invention;

FIG. 6: schematic illustration of an implant system, incorporating two intracorporeal devices, in accordance with an embodiment of the present invention;

FIG. 7: schematic illustration of a self-expansible anchor having a carrier for mounting an intracorporeal device thereon, in accordance with an embodiment of the present invention;

FIG. 8: schematic illustration of a self-expansible structure, operable as a stent, in accordance with an embodiment of the present invention;

FIGS. 9A-9B: schematic illustrations of a retrieval system and an implant system adapted for retrieval, in accordance with an embodiment of the present invention;

FIGS. 10A-10B: schematic illustrations of symmetry considerations of implant systems; and

FIGS. 11A-11B: schematic illustrations of sizing considerations for implant systems.

### DESCRIPTION OF THE PREFERRED EMBODIMENTS

The present invention is of a self-expansible structure for implantation in a body cavity. The structure has a generally tubular outline, a nominal length, a nominal diameter, and a unique behavior under constraint. The unique behavior is expressed by a transition diameter, at which a constrained behavior of the structure changes so that, at a constraining diameter smaller than the transition diameter, the structure conforms to the constraint by decreasing the structure's diameter, to below the nominal diameter, and elongating beyond the nominal length, while at a constraining diameter larger than the transition diameter but smaller than the nominal diameter the structure conforms to the constraint by decreasing the structure's diameter below the nominal diameter, while substantially maintaining the nominal length. Thus, the structure is operable for insertion into a body cavity, via a catheter, having a catheter inner diameter smaller than the transition diameter, by decreasing the structure's diameter, and elongating, and the structure is operable for implantation in the body cavity, having a cavity inner diameter greater than the transition diameter but smaller than the nominal diameter, by decreasing the structure's nominal diameter, while substantially maintaining the nominal length. The value of the transition diameter may be varied and controlled by the manner of constraining in the catheter, during insertion.

In the preferred embodiment, the structure is formed of two closed wire constructions, shaped as symmetric wings, and has no exposed tips. The structure may be a stent. Alternatively, the structure may be an anchor, for mounting an incorporeal device thereon.

The principles and operation of the system and method according to the present invention may be better understood with reference to the drawings and accompanying descriptions.

Before explaining embodiments of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein are for the purpose of description and should not be regarded as limiting.

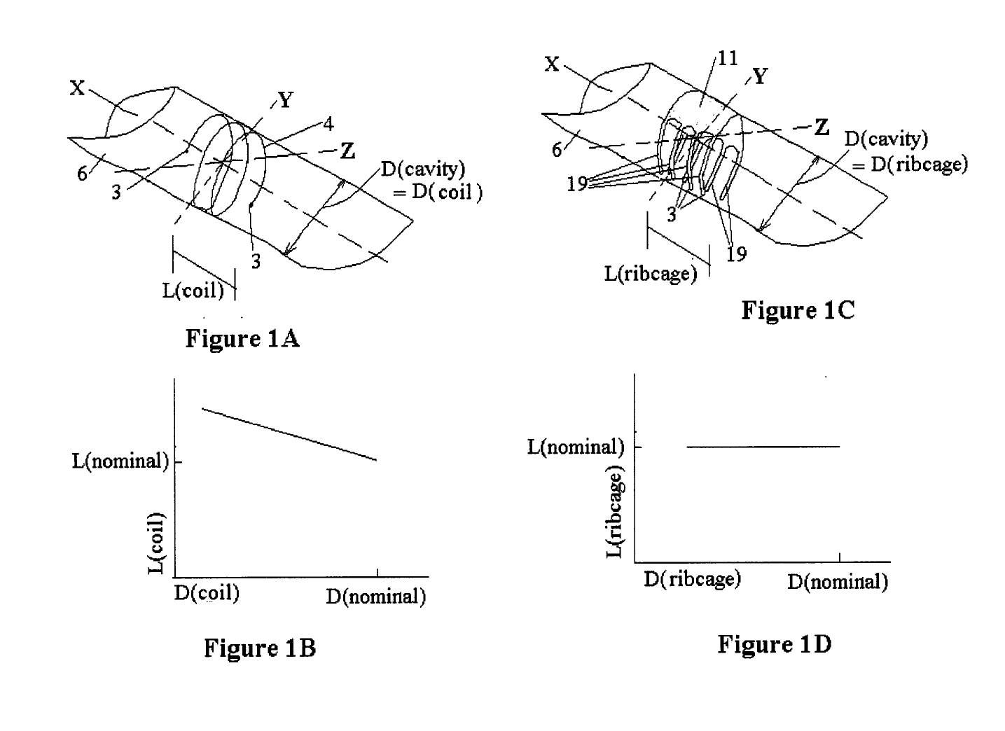

Referring now to the drawings, FIGS. 1A-1D schematically illustrate relationships between length and diameter of prior-art, self-expansible stents, operable to adjust to the size of a cavity in which they are deployed.

A first self-expansible stent of the prior art, seen in FIG. 1A, is a coil stent 4, similar for example, to a self-expanding Nitinol coil stent known as the Coronary Cardiocoil, developed by Mederonic InStent. (The Handbook of Coronary Stents Rotterdam Thoraxcenter Group, Edited by P. W. Serruys and M. J. B. Kutryk, 2nd edition, 1998, Chapter 25, by Rafael Beyar, pp. 243-249.)

Self-expansible coil stent 4, having tips 3, may adjust to the size of a body cavity 6; when deployed, the deployed coil diameter, D(coil) substantially equals the cavity size, D(cavity), provided the cavity size is somewhat smaller than the coil nominal diameter, D(nominal). However, when deployed, the coil length L(coil) is greater than the coil nominal length L(nominal). For coil stent 4, there is an inverse relationship between D(coil) and L(coil), as seen in FIG. 1B.

Since the size of the cavity is not precisely known, the actual length of deployed coil stent 4 is also not precisely known. This may cause problems in situations where space is tight, and it is desired to know the precise length of deployed coil stent 4.

A second self-expansible stent of the prior art, seen in FIG. 1C, is a ribcage-like stent, similar for example, to that taught by U.S. Pat. No. 6,273,908, to Ndondo-Lay, whose disclosure is incorporated herein by reference.

Self-expansible ribcage-like stent 11, having appendages 19 and tips 3, may adjust to the size of body cavity 6, provided it is somewhat smaller than the nominal diameter, D(nominal) of ribcage-like stent 11. Thus, in the deployed condition, the deployed diameter D(ribcage) substantially equals the cavity size, D(cavity). At the same time, a length L(ribcage) remains substantially at the nominal value L(nominal) for ribcage-like stent 11, as seen in FIG. 1D. This feature is of importance, for evaluating the effect of stent 11 in cavity 6.

However, for inserting a stent to a body cavity, via a catheter, coil stent 4 is preferred to ribcage-like stent 11, for the following reasons:

1. For insertion into a catheter, coil stent 4 may be elongated, to a straight wire. On the other hand, ribcage-like stent 11, must be coiled to a dense spiral, somewhat like pastry dough, to maintain the nominal length. Technically, it is simpler to insert a structure into a catheter by elongating it than by coiling it into a dense spiral. In fact, coiling ribcage-like stent 11 to a dense spiral, evenly along its length, may require a special instrument, a requirement not necessary for coil stent 4.

2. Additionally, when constrained in a catheter, coil stent 4, being elongated, remains relatively flexible and does not hinder the maneuvering of the catheter through the blood vessels. However, ribcage-like stent 11, having been densely coiled, is relatively stiff, posing a hindrance to the maneuverability of the catheter.

Thus, when comparing the features of coil stent 4 and ribcage-like stent 11, one may conclude that although when deployed in the body cavity, the substantially constant-length feature of ribcage-like stent 11 is the more advantageous, for insertion into the body cavity, via a catheter, the inverse relationship between length and diameter of coil stent 4 is preferred.

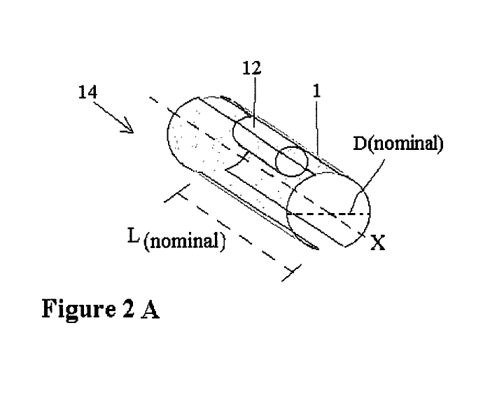

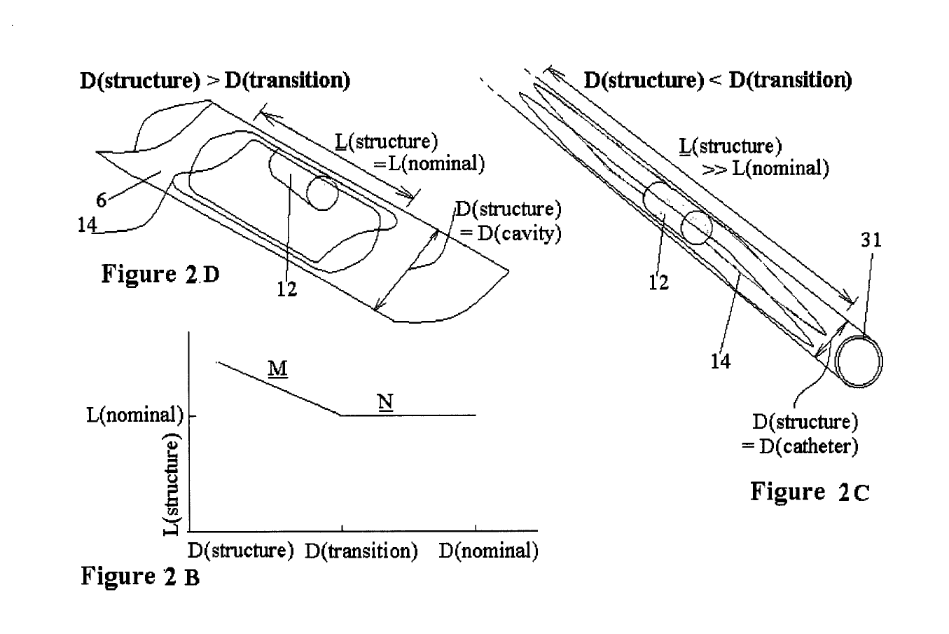

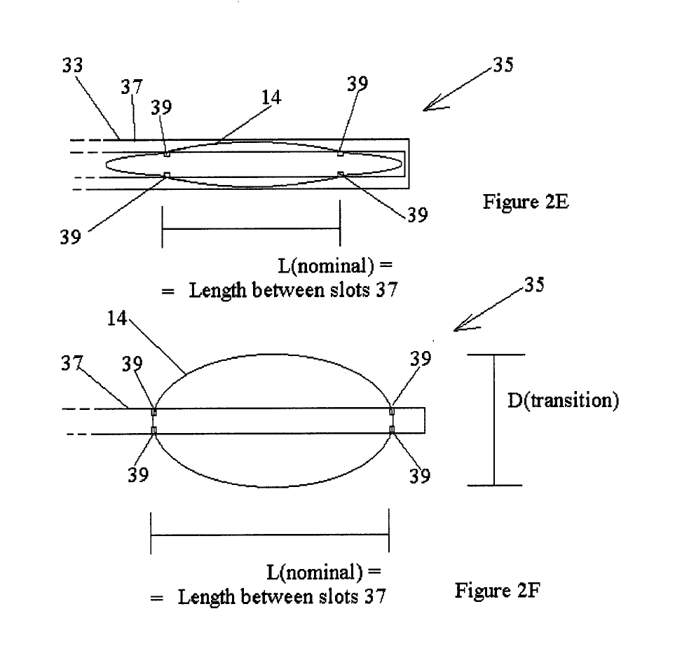

With this conclusion in mind, reference is now made to FIGS. 2A-2F, which schematically illustrate a self-expansible structure 14, in accordance with the present invention.

As seen in FIG. 2A, self-expansible structure 14 has a generally tubular outline 1, a nominal length L(nominal), and a nominal diameter D(nominal). The nominal length, L(nominal), may be defined as the length between the extreme terminal points of self-expansible structure 14, when in a fully relaxed state. The nominal diameter, D(nominal), may be defined as the diameter of the smallest factionary ring, through which self-expansible structure 14 may be positioned, when in a fully relaxed state.

Self-expansible structure 14 is adapted to conform to a generally tubular constraint, for example, a blood vessel 6, another body cavity 6, or a catheter 31, when inserted thereto, and has a transition diameter, D(transition) at which a constrained behavior of self-expansible structure 14 changes. At a constraining diameter smaller than D(transition), self-expansible structure 14 conforms to the constraint by decreasing the structure's diameter, to below the nominal diameter, and elongating beyond the nominal length, while at a constraining diameter larger than D(transition), but smaller than the nominal diameter, self-expansible structure 14 conforms to the constraint by decreasing the structure's diameter below the nominal diameter, while substantially maintaining the nominal length.

This behavior is illustrated in FIG. 2B. When constrained in a tube of a diameter smaller than D(transition), structure 14 behaves in accordance with a portion M of the curve, displaying an inverse relationship between the constrained diameter and the constrained length. But when constrained in a tube of a diameter larger than D(transition), but smaller than D(nominal), structure 14 behaves in accordance with a portion N of the curve, displaying a substantially constant length. The variable length and diameter of self-expansible structure 14 may be referred to as L(structure) and D(structure).

As seen in FIG. 2C, self-expansible structure 14 is operable for insertion into a body cavity 6, via a catheter 31 having a catheter inner diameter smaller than D(transition), by decreasing the structure's diameter and elongating, and

As seen in FIG. 2D, self-expansible structure 14 is operable for implantation in the body cavity 6, having an inner cavity diameter greater than D(transition), but smaller than D(nominal), by decreasing the structure's diameter, while substantially maintaining the nominal length.

Thus, self-expansible structure 14 combines the advantages of ribcage-like stent 11 and of coil stent 4. On the one hand, self-expansible structure 14 remains at the nominal length, when deployed, in a manner similar to ribcage-like stent 11. On the other hand, it provides ease of insertion into a catheter, and resiliency and flexibility when maneuvering the catheter along a guide wire, in a manner similar to coil stent 4.

It will be appreciated that the transition diameter is an inherent feature of self-expansible structure 14 of the present invention, being a consequence of the material and its unique geometry. However, the value of the transition diameter may be varied and controlled by the manner of constraining in the catheter, during deployment. In accordance with a preferred embodiment of the present invention, deployment is carried out as a two-stage expansion process, as taught by commonly owned US Patent application, “Deployment Device, System and Method for Medical Implantation,” to Fastovsky, V. et al., filed concurrently with the present application and incorporated herein by reference. In particular, a portion of self-expansible structure 14 is wrapped, coiled around, hooked, inserted into slots, or otherwise fixed, within the deployment device. The first stage of expansion includes contraction to the nominal length, while still on the deployment device. The second stage of expansion, after release from the deployment device, includes radial expansion, at the nominal length.

Accordingly, as seen in FIG. 2E, when constrained in a deployment device 35, self-expansible anchor 14 is arranged between an outer tube 33 and an inner tube 37, while fixed onto inner tube 37, for example, by at least two slots 39. Preferably, the distance between slots 39 is substantially the same as the nominal length, L(nominal) of self expansible structure 14. It will be appreciated that the inner diameter of outer tube 33 defines the catheter inner diameter, hereinabove (FIG. 2C).

As seen in FIG. 2F, The first stage of expansion occurs when outer tube 33 is withdrawn. Self-expansible strucutre 14 then expands while contracting to its nominal length. The diameter that is reached directly when the nominal length is reached, is defined as the transition diameter D(transition).

Self-expansible strucuture 14 expands further, substantially to the diameter of the cavity at that location, while at the nominal length. Inner tube 37 is then withdrawn.

Structure 14 may be a stent. Alternatively, it may be an anchor for mounting an incorporeal device 12 thereon.

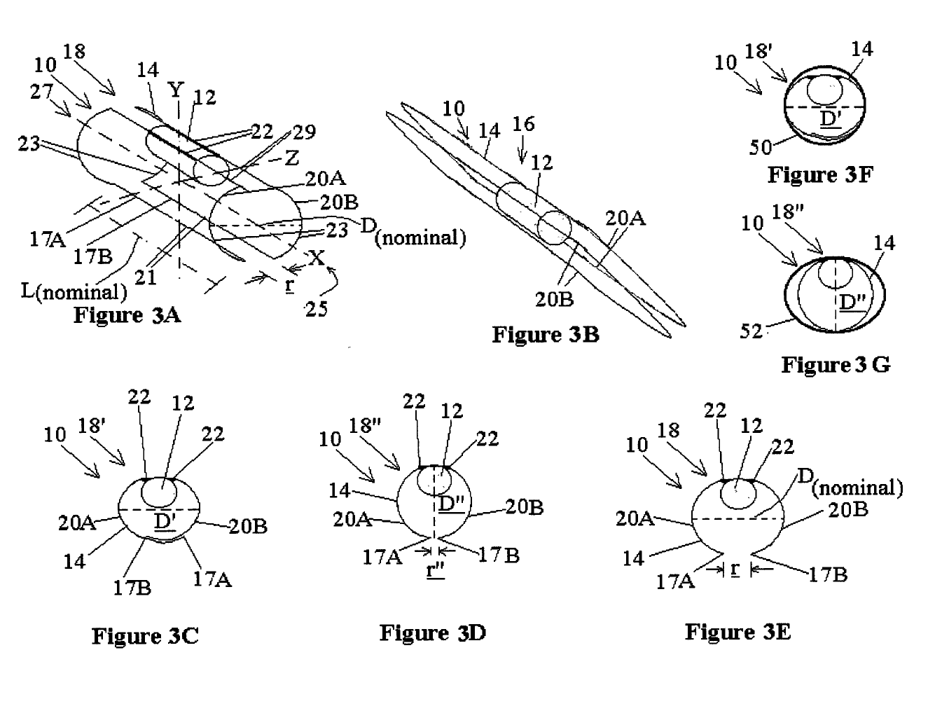

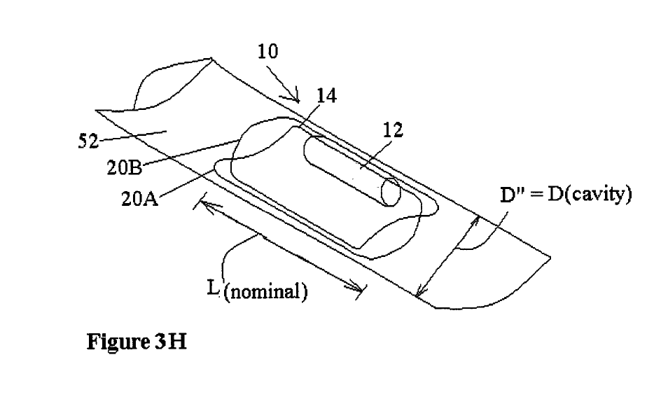

Referring further to the drawings, FIGS. 3A-3H schematically illustrate an implant system 10, comprising self-expansible structure 14, operative as self-expansible anchor 14, according to a preferred embodiment of the present invention.

As seen in FIG. 3A, implant system 10 defines an X; Y; Z coordinate system, referenced to its center point. In a blood vessel, implant system 10 is preferably disposed with the X-axis along the direction of blood flow. Implant system 10 further defines a first end 25 and a second end 27.

Additionally, implant system 10 includes at least one intracorporeal device 12, mounted on self-expansible anchor 14. For example, intracorporeal device 12 may be welded, crimped, or bonded onto self-expansible anchor 14, along lines 22.

In a fully expanded, relaxed condition 18, seen in FIG. 3A, self-expansible anchor 14 includes at least one, and preferably two first length wires 29 and two second length wires 21, all of lengths L(nominal), connecting C-shape edges 23. Together, they form two closed wire constructions 20A and 20B, disposed as two closed wire constructions, outlining a tubular space within, and defining at least one fully expanded, relaxed size D(nominal). Together, two closed wire constructions 20A and 20B form self-expansible anchor 14. Preferably, closed wire constructions 20A and 20B are symmetric. Closed wire constructions 20A and 20B may be thought of as symmetric wings, attached to intracorporeal device 12.

It will be appreciated that while each of wire constructions 20A and 20B is a closed structure, overall, self-expansible anchor 14 has an open structure.

The open structure is realized by a closure distance r, between edges 17A and 17B. In fully expanded, relaxed condition 18, edges 17A and 17B of closed wire constructions 20A and 20B define a fully expanded, relaxed closure distance r between them.

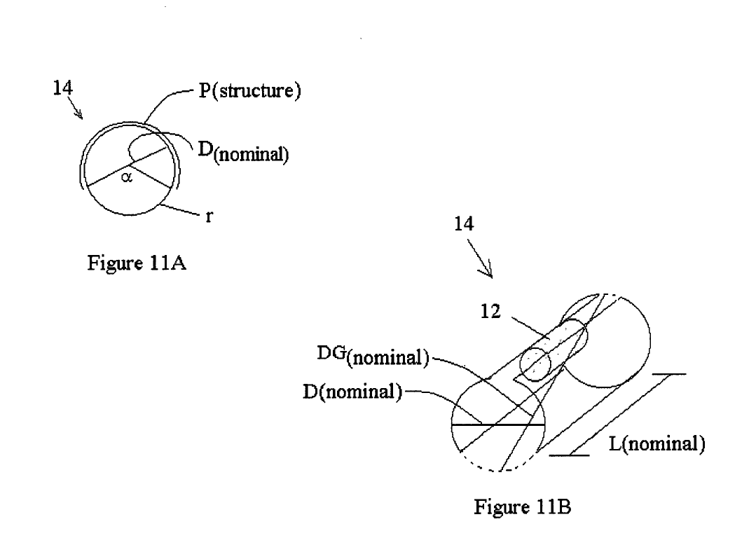

The purpose of first and second length wires 29 and 21 is to lengthen implant system 10, beyond the length of intracorporeal device 12, to a minimal length required for stability, when necessary. While the size of the cavity may be as large as 40 mm, the length of intracorporeal device 12 may be only 10-20 mm. Were implant system 10 limited to the length of intracorporeal device 12, an unstable situation would arise. Stability considerations dictate that nominal length L(nominal) of implant system 10 be at least 25% greater than the nominal diameter D(nominal). Alternatively, as will be illustrated in conjunction with FIG. 11B, hereinbelow, the nominal diagonal of implant system 10 has to be at least 25% greater than the nominal diameter D(nominial). Additionally, the purpose of length wires 21 is to avert exposed tips, which could scratch or injure the cavity walls.

Furthermore, length wires 21 prevent C-shapes sides 23 of the left and right sides from shifting with respect to each other, so as to create bending moments on the cavity walls, and (or) to extend the length of anchor 14.

The expansion of self-expansible anchor 14 is illustrated hereinbelow, in FIGS. 3C-3E, viewed from the X-direction, wherein edges 17A and 17B may overlap, touch, or be apart, depending on the extent of expansion.

In a contracted condition 16, seen in FIG. 3B, self-expansible anchor 14 is adapted in size and shape for insertion via a catheter, as closed wire constructions 20A and 20B are elongated.

In an expanded condition 18′, seen in FIG. 3C, edges 17A and 17B overlap, defining an expanded size D′ smaller than D(nominal).

In another expanded condition 18″, seen in FIG. 3D, edges 17A and 17B define an expanded closure distance r″ smaller than r (FIG. 3A) and an expanded size D″ smaller than D(nominal), but possibly larger than expanded size D′.

In fully expanded, relaxed condition 18, further illustrated in FIG. 3E, closed wire constructions 20A and 20B define fully expanded, relaxed size D(nominal) while edges 17A and 17B define fully expanded, relaxed closure distance r.

It will be appreciated that many intermediate levels of expansions are similarly possible.

As seen in FIG. 3F, in a cavity 50, self-expansible anchor 14 gradually expands to size D′ of expanded condition 18′ (FIG. 3C), operative for anchoring.

As seen in FIG. 3G, in a cavity 52, self-expansible anchor 14 gradually expands to size D″ of expanded condition 18″ (FIG. 3D), operative for anchoring.

In accordance with the present invention, while the diameter of implant system 10 may vary from D(nominal) to D′ or D″, the length of implant system 10 remains substantially as L(nominal).

It will be appreciated that C-shape sides 23 of closed wire constructions 20A and 20B (FIG. 3A) may form elliptical or circular shapes. Additionally, C-shape sides 23 may alternate between circular and elliptical shapes, or between different orientations of elliptical shapes during the expansion, as seen in FIGS. 3C-3E. Similarly, the cavity may be circular or elliptical. For the present discussion, an operative anchor size is that which is oriented in a direction that forms anchorage with the walls of the cavity, as seen in FIGS. 3F-3G.

Additionally, it will be appreciated that, where the cavity size changes along length of implant system 10, the extent of expansion may vary between first end 25 and second end 27. Additionally, it will be appreciated that some adjustments may be made by the expanded sizes and the walls of the cavities, as they arrive to equilibrium operative conditions.

FIG. 3H is a pictorial representation of the embodiment of FIG. 3G, illustrating cavity 52, such as a blood vessel 52, having a cavity size D(cavity). Implant system 10, anchored therein, is at expanded condition 18″, having expanded diameter D″ and length L(nominal).

Structure 14 is designed to exert a pressure of between 5 and 300 mm Hg on walls of the cavity. Additionally, the integral of (pressure)×(contact area) is designed to be in the range of 1-30 gmf, and preferably, between 5 and 25 gmf.

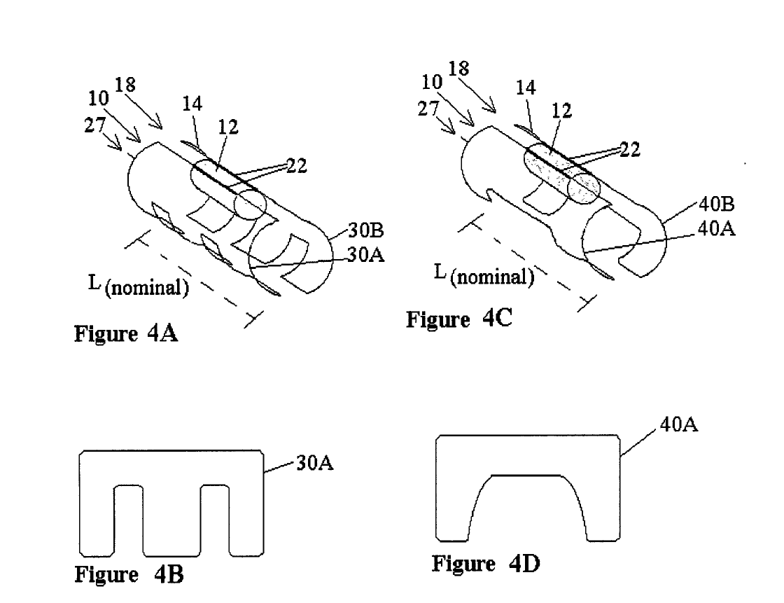

Referring further to the drawings, FIGS. 4A-4D schematically illustrates implant system 10, comprising self-expansible anchor 14, according to other embodiments of the present invention.

As seen in FIGS. 4A and 4B, implant system 10 includes intracorporeal device 12, mounted on self-expansible anchor 14, comprising two closed wire constructions 30A and 30B, similar to closed wire constructions 20A and 20B of FIG. 3A, but designed for somewhat increased contact area with the cavity walls, for greater stability.

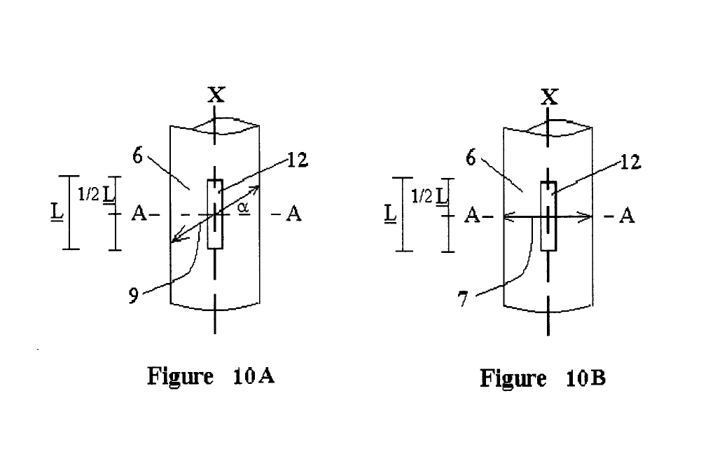

It will be appreciated that two closed wire constructions 30A and 30B are somewhat similar to, or can be made similar to ribcage-like stent 11 of FIG. 1C, hereinabove, (based on U.S. Pat. No. 6,273,908, to Ndondo-Lay). Wire constructions 30A and 30B may be thought of as a skeleton and appendages However, in accordance with the present invention, closed wire constructions 30A and 30B behave in accordance with FIG. 2B hereinabove, and have no exposed tips. Preferably, wire constructions 30A and 30B are symmetric. Alternatively, they are arranged to intertwine, but maintain a symmetry with respect to a plane A-A, of FIGS. 10A and 10B, hereinabove. (In other words, the intertwining is mirrored across plane A-A).

As seen in FIGS. 4C and 4D, implant system 10 includes intracorporeal device 12, mounted on self-expansible anchor 14, composing two closed wire constructions 40A and 40B, similar to closed wire constructions 30A and 30B of FIG. 4A, but having a somewhat different geometry.

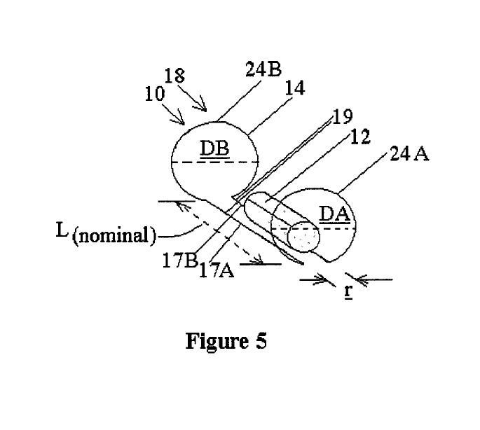

Referring further to the drawings, FIG. 5 schematically illustrates implant system 10, comprising self-expansible anchor 14, according to still another preferred embodiment of the present invention. Implant system 10 includes intracorporeal device 12, mounted on self-expansible anchor 14. In accordance with the present embodiment, self-expansible anchor 14 is formed of a single wire construction, shaped as two open loops 24A and 24B, connected by two length wires 19, adapted for pressing against the cavity walls and anchoring therein.

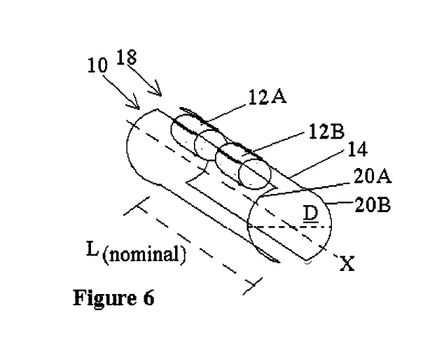

Referring further to the drawings, FIG. 6 schematically illustrates implant system 10, comprising self-expansible anchor 14, according to yet another preferred embodiment of the present invention, wherein implant system 10 includes two intracorporeal device 12A and 12B, mounted on self-expansible anchor 14.

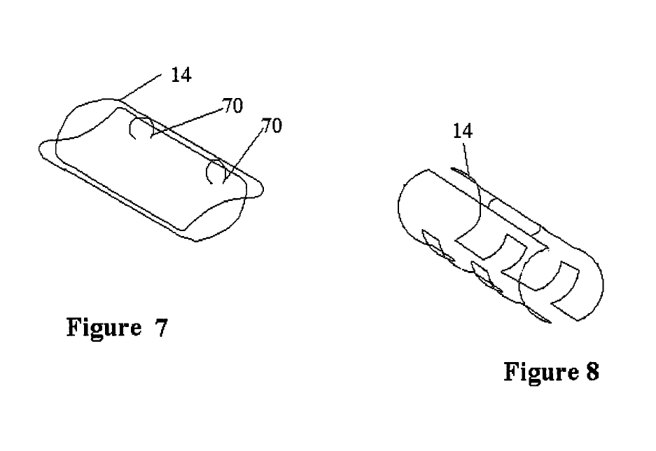

Referring further to the drawings, FIG. 7 schematically illustrates the self-expansible anchor 14 having at least one carrier 70, for mounting the intracorporeal device 12 thereon. Thus, self-expansible anchor 14 may be provided as such, and any one of a variety of the intracorporeal devices 12 may be fitted onto at least one carrier 70, later on.

Referring further to the drawings, FIG. 8 schematically illustrates self-expansible structure 14, operable as a stent.

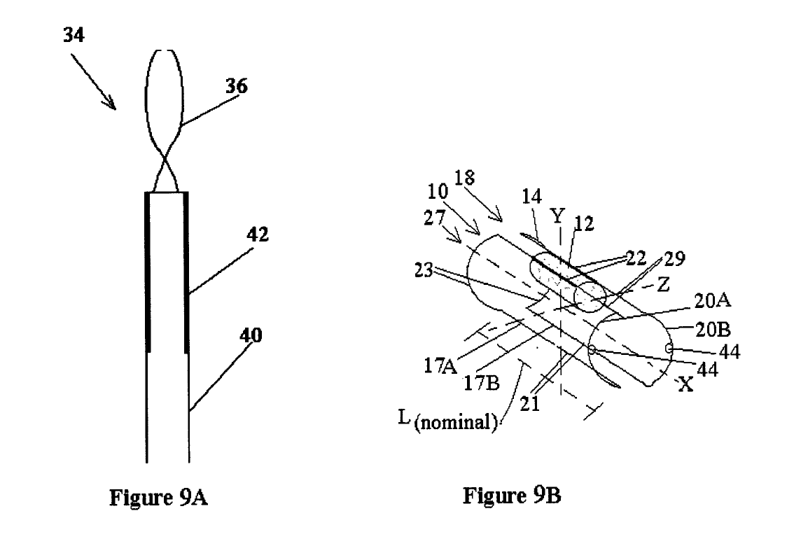

Referring further to the drawings, FIGS. 9A-9B schematically illustrate a retrieval instrument 34 and implant system 10 adapted for retrieval, according to a preferred embodiment of the present invention.

As seen in FIG. 9A, retrieval instrument 34 includes a gripping device 36, arranged inside a catheter 40, which preferably includes a reinforced distal edge 42, with respect to an operator (not shown). Gripping device 36 may be a clamp, adapted to be manipulated extracorporeally, or a hook.

As seen in FIG. 9B, self-expansible structure 14 may further include an inner component 44 for engaging with a retrieval instrument. Inner component 44 may be at least one, and preferably two inner loops 44, adapted to be gripped by gripping device 36 (FIG. 9A). Gripping device 36 grips and pulls inner structure 44 into reinforced distal edge 42, and implant system 10 may be retrieved by catheter 40.

Referring again to FIGS. 1A and 1C and to FIGS. 2B-9B, additional advantages of the present invention, over prior art systems may be observed.

As seen in FIGS. 1A and 1C, the two self-expansible stents of the prior art have exposed tips 3, which may scratch or injure the tissue. By contrast, self-expansible structure 14 of the present invention has no exposed tip, being formed of closed wire constructions.

Additionally, as seen in FIGS. 1A and 1C, the two self-expansible stents of the prior art are deployed asymmetrically—the appendages of ribcage-like stent 11 are shifted to respect with each other, so as to intertwine, while spirals such as coil stent 4 are inherently asymmetric. In consequence, bending moments may be generated in the prior art systems and may lead to toppling.

Referring further to the drawings, FIGS. 10A and 10B further evaluate and compare the asymmetry of prior art system with the symmetry of the present invention.

FIG. 10A schematically illustrates a line of action 9 of coil stent 4 or of ribcage-like stent 11 on the walls of cavity 6. Line of action 9 is at an angle α to the X axis. A bending moment may be generated when there is asymmetry with respect to a plane A-A, orthogonal to the X axis and crossing the X axis at the midlength of the implant system, as shown in FIG. 10A.

FIG. 10B schematically illustrates a line of action 7 of embodiments of the present invention. Line of action 7 is orthogonal to the X axis. There is symmetry with respect to plane A-A, and no bending moment is generated.

Referring further to the drawings, FIGS. 11A and 11B are schematic illustrations of sizing considerations for implant system 10.

FIG. 11A illustrates self-expansible structure 14 on the forming mandrel. Self-expansible structure 14 has a perimeter, P(structure), while an angle α defines closure arc, r, in radians, in the fully relaxed condition. We may write a relationship between P(structure) and the nominal diameter, D(nominal), as follows:

P(structure)=(π-α/2) D(nominal) [1]

Defining D(upper limit) as the maximum deployed diameter for self expansible structure 14, and D(lower limit) as the minimum deployed diameter for self expansible structure 14, there must always be some oversizing, or:

D(nominal)>D(upper limit)>D(lower limit). [2]

In accordance with the present invention, in the deployed state, P(structure) should be at least as great as half the cavity perimeter, and preferably, at least as great as two thirds the cavity perimeter. This is so that sufficient anchorage against the cavity walls takes place. Thus,

P(structure)≧½π D(upper limit), [3]

and preferably,

P(structure)≧⅔ π D(upper limit). [4]

When overlap is not permitted, an additional restriction is given by:

P(structure)≦π D(lower limit). [5]

In accordance with the present invention, overlap may occur, but in accordance with the preferred embodiment, overlap is avoided, and r≧0.

Additionally, as seen in FIG. 11B, stability considerations dictate that nominal length L(nominal) be at least 25% greater than the nominal diameter D(nominal). Alternatively, the nominal diagonal DG (nominal) has to be at least 25% greater than the nominal diameter D(nominal).

### EXAMPLE

Reference is now made to the following examples, seen in Table 1, which together with the above descriptions, illustrate the invention in a nonlimiting fashion.

TABLE 1

| | D (nominal) | D (upper- | D (lower- | α | P (structure) |
|---|---|---|---|---|---|
| | mm | limit) mm | limit) mm | radians | mm |
| Without | 25 | 21 | 15 | 5π/6 | ˜45.8 |
| overlap | | | | | |
| With | 25 | 21 | 12-14 | 5π/6 | ˜45.8 |
| overlap | | | | | |
| Without | 20 | 18 | 13 | 3π/4 | ˜39.3 |
| overlap | | | | | |
| With | 20 | 18 | 10-12 | 3π/4 | ˜39.3 |
| overlap | | | | | |

In accordance with the present invention, self-expansible structure 14 is formed of a shape memory alloy. In accordance with a first preferred embodiment, self-expansible structure 14 is fully austenitic at room temperature, and above. In accordance with a second preferred embodiment, self-expansible structure 14 is fully austenitic at body temperature and above. Preferably, self-expansible structure 14 further comprises a stress-induced martensite phase at the fully austenitic temperature range.

Self-expansible structure 14 may be constrained in catheter 31 (FIG. 2C), for insertion into the body cavity, at a martensite phase, at a martensite temperature range. Alternatively, self-expansible structure 14 may be constrained in a catheter, at a stress induced martensite phase, at the fully austenitic temperature range.

Additionally, self-expansible structure 14 may be operational in the stress-induced martensite phase, when deployed, or in the in the asutentic phase, when deployed, both at the fully austenitic temperature range.

In accordance with the present invention, the wire diameter of closed wire structures such as 20A and 20B (FIG. 3A), 30A and 30B (FIG. 4A), or 40A and 40B (FIG. 4C) is between 0.03 and 1.0 mm. Preferably, the wire diameter is between 0.2 and 0.3 mm. It will be appreciated that other values are similarly possible.

In accordance with the present invention, the body cavity may be a blood vessel, a chamber of a heart, a ventricle of a heart, an airway passage, a uterus, a bowl, a digestive tract, or any other body cavity or lumen.

Furthermore, in accordance with the present invention, the body cavity may be a human body cavity or an animal body cavity.

Additionally, in accordance with the present invention, intracorporeal device 12 comprises at least one device, for example, of the following: a pressure sensor, a flow rate sensor, a temperature sensor, an oxygen concentration sensor, an ion concentration sensor, an impedance sensor, a sensor adapted for cardiac output assessment, a filter, such as a blood filter, a septal occluder, a coil, a detachable coil for aneurysm treatment, a graft, a deflector, and any other device for performing or measuring a physiological function or parameter within a body cavity.

Furthermore, in accordance with the present invention, intracorporeal device 12 may comprise a plurality of sensors and (or) devices.

In accordance with the present invention, intracorporeal device 12 may be an energizeable device. Specifically, intracorporeal device 12 may be acoustically energizeable, and include an acoustic transducer, such as a piezoelectric transducer. Alternatively, or additionally, intracorporeal device 12 may be electromagnetically energizeable and include a ferroelectric element. Alternatively or additionally, intracorporeal device 12 may be magnetically energizeable and include a magnet. Alternatively, or additionally, intracorporeal device 12 may be radio frequency energizeable and include a radio frequency antenna or coil and a capacitor.

For example, intracorporeal device 12 may be that described in commonly owned U.S. patent application Ser. No. 09/872,129 (Publication No. 20010026111), to Doron et al., “Acoustic biosensor for monitoring physiological conditions in a body implantation site,” incorporated herein by reference. U.S. patent application Ser. No. 20010026111 describes an acoustic biosensor for deployment at an implantation site within a body, such as an abdominal aortic aneurysm. The biosensor includes a sensor element for measuring a physiological condition at the implantation site, and for generating an information signal representative of the physiological condition. The biosensor further includes a piezoelectric transducer element for converting an externally originated acoustic interrogation signal into energy for operating the sensor, and for modulating the interrogation signal, e.g., by employing a switching element to alternate the mechanical impedance of the transducer element, to transmit the information signal outside of the body.

Additionally or alternatively, intracorporeal device 12 may include a piezoelectric transducer, described in commonly owned U.S. Pat. No. 6,140,740 to Porat, et al “Piezoelectric transducer,” incorporated herein by reference. U.S. Pat. No. 6,140,740 describes a miniature piezoelectric transducer element, comprising; (a) a cell element having a cavity; (b) a flexible piezoelectric layer attached to the cell member, the piezoelectric layer having an external surface and an internal surface, the piezoelectric layer featuring such dimensions so as to enable fluctuations thereof at its resonance frequency upon impinging of an external acoustic wave; and (c) a first electrode attached to the external surface and a second electrode attached to the internal surface of the piezoelectric layer. At least one of the electrodes may be specifically shaped so as to provide a maximal electrical output, wherein the electrical output may be current, voltage or power. A preferred shape of the electrodes includes two cores interconnected by a connecting member. The transducer element may function as a transmitter. When used as a transmitter, the electrodes are electrically connected to an electrical circuit including a switching element for modulating the reflected acoustic wave by controllably changing the mechanical impedance of the piezoelectric layer according to the frequency of an electrical message signal arriving from an electronic member, such as a sensor. Third and fourth electrodes may be attached to the piezoelectric layer and the electrical circuit, such that the switching element alternately connects the electrodes in parallel and anti-parallel electrical connections so as to controllably change the mechanical impedance of the piezoelectric layer.

Furthermore, intracorporeal device 12 may be that described in commonly owned U.S. Pat. No. 6,277,078 to Porat, et al, “System and method for monitoring a parameter associated with the performance of a heart,” incorporated herein by reference. U.S. Pat. No. 6,277,078 describes an intrabody implantable system for long-term, real time monitoring of at least one parameter associated with heart performance. The system includes (a) a first sensor being implantable within a heart and being for collecting information pertaining to a pressure in a first cavity of the heart; (b) at least one additional sensor being implantable in a blood vessel supporting blood flow into or out of a second cavity of the heart, the at least one additional sensor being for collecting information pertaining to a pressure and a flow within the blood vessel; and (c) at least one device implantable in the body and being in data communication with the first sensor and the at least one additional sensor, the at least one device being for receiving the information pertaining to the pressure in the first cavity of the heart and the information pertaining to the pressure and the flow within the blood vessel and for relaying the information pertaining to the pressure in the first cavity of the heart and the information pertaining to the pressure and the flow within the blood vessel outside the body.

In accordance with U.S. Pat. No. 6,277,078, the at least one device includes at least one transducer for converting electric signal into a radiative signal, wherein the radiative signal is selected from the group consisting of radio frequency, a magnetic field, an electric field and acoustic radiation. For example, the at least one transducer may be an acoustic transducer and the radiative signal may be an acoustic signal. Alternatively, the at least one transducer may be a magnetic field transducer and the signal may be a magnetic field signal.

Additionally, in accordance with U.S. Pat. No. 6,277,078, the system may include at least one power source, preferably integrated into the at least one device and preferably, arranged as an at least one energizeable power source. The at least one energizeable power source includes at least one transducer for converting a radiative energy into electric energy.

Furthermore, in accordance with U.S. Pat. No. 6,277,078, the radiative energy for energizing the energizeable power source is selected from the group consisting of radio frequency, a magnetic field, an electric field and acoustic radiation. For example, the at least one transducer may be an acoustic transducer and the radiative energy may be an acoustic energy. Alternatively, the transducer may be a magnetic field transducer and the radiative energy may be a magnetic field.

Additionally, intracorporeal device 12 may be that described in commonly owned U.S. Pat. No. 6,237,398 to Porat, et al., “System and method for monitoring pressure, flow and constriction parameters of plumbing and blood vessels,” incorporated herein by reference. U.S. Pat. No. 6,237,398 describes a system and method of quantifying flow, detecting a location of an obstruction and quantifying a degree of the obstruction in a pipe characterized in pulsatile flow. The method includes the steps of (a) attaching at least two spaced pressure sensors onto inner walls of the pipe; (b) using the at least two spaced pressure sensors for recording pressure records associated with each of the at least two pressure sensors within the pipe; and (c) using the pressure records for quantifying the pulsatile flow in the pipe, for detecting the location of the obstruction in the pipe and for quantifying the degree of the obstruction in the pipe.

In accordance with the present invention, intracorporeal device 12 may be capable of telemetric communication with an extracorporeal device.

For example, intracorporeal device 12 may be that described in commonly owned U.S. Pat. No. 6,198,965 to Penner, et al., “Acoustic telemetry system and method for monitoring a rejection reaction of a transplanted organ,” incorporated herein by reference. U.S. Pat. No. 6,198,965 describes a telemetry system for monitoring a rejection reaction of a transplanted organ being transplanted within a patient's body. The telemetry system includes (a) a telemetry control unit located outside the body of the patient; and (b) a telemetry monitoring unit implanted within the body of the patient, the telemetry monitoring unit including: (i) at least one acoustic transducer being for receiving an acoustic signal from the telemetry control unit and converting the acoustic signal into a first electrical signal, the at least one acoustic transducer further being for receiving a second electrical signal and converting the second electrical signal into a transmitted acoustic signal receivable by the telemetry monitoring unit; and (ii) a plurality of electrodes positionable in intimate contact with, or deep within, the transplanted organ and being in communication with the at least one acoustic transducer, the plurality of electrodes being for passing the first electrical signal through the transplanted organ for monitoring the electrical impedance thereof and further being for relaying the second electrical signal corresponding to the electrical impedance to the at least one acoustic transducer so as to enable the monitoring of the presence or absence of the rejection reaction.

Additionally, intracorporeal device 12 may be that described in commonly owned U.S. Pat. No. 6,239,724, to Doron et al., “System and method for telemetrically providing intrabody spatial position,”incorporated herein by reference. U.S. Pat. No. 6,239,724 describes a telemetry system and method for providing spatial positioning information from within a patient's body. The system includes at least one implantable telemetry unit which includes (a) at least one first transducer being for converting a power signal received from outside the body, into electrical power for powering the at least one implantable telemetry unit; (b) at least one second transducer being for receiving a positioning field signal being received from outside the body; and (c) at least one third transducer being for transmitting a locating signal transmittable outside the body in response to the positioning field signal.

Furthermore, in accordance with the present invention, implant system 10 is adapted for insertion in accordance with the teaching of commonly owned US Patent application, “Deployment Device, System and Method for Medical Implantation,” to Fastovsky, V. ct al., filed concurrently with the present application, and incorporated herein by reference.

The deploying system may thus comprise: an inner tube; an outer tube; and an implant system received on the inner tube and enclosed by the outer tube. The implant system includes an implantable device, mounted on an adjustable, self-expansible anchor which is in a contracted condition when enclosed by the outer tube. The adjustable, self-expansible anchor expands to a partially-expanded condition when the outer tube is retracted, and to a fully-expanded condition when the inner tube is removed together with the outer tube. The implant system is deployed by introducing the deployment device to the target location in the body cavity; retracting the outer tube with respect to the implant system such that the anchor self-expands from its contracted condition to its partially-expanded condition; and withdrawing the inner tube from the implant system such that the anchor self-expands from its partially-expanded condition to its fully-expanded condition to firmly fix the implant system at the target location within the body cavity.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A self-expansible structure, for implantation in a body cavity, said structure comprising:
a generally tubular outline;
a nominal length;
a nominal diameter; and
a transition diameter, at which a constrained behavior of said structure changes so that:
at a constraining diameter smaller than said transition diameter, said structure conforms to the constraint by decreasing the structure's diameter, to below the nominal diameter, and elongating beyond the nominal length, and
at a constraining diameter larger than said transition diameter but smaller than the nominal diameter, said structure conforms to the constraint by decreasing the structure's diameter below the nominal diameter, while substantially maintaining the nominal length,
wherein:
said structure is operable for insertion into a body cavity, via a catheter, having a catheter inner diameter smaller than said transition diameter, by decreasing the structure's diameter, while elongating, and
said structure is operable for implantation in the body cavity, having a cavity inner diameter greater than said transition diameter but smaller than the nominal diameter, by decreasing the structure's nominal diameter, while substantially maintaining the nominal length.

2. The self-expansible structure of claim 1, wherein said structure is a stent, designed to provide structural support to a blood vessel.

3. The self-expansible structure of claim 1, wherein said structure is an anchor, designed to support an intracorporeal device thereon.

4. The self-expansible structure of claim 3, wherein said anchor further includes an intracorporeal device, mounted thereon, adapted for performing a physiologically related task at the body cavity.

5. The self-expansible structure of claim 4, wherein said intracorporeal device is selected from the group consisting of a pressure sensor, a flow rate sensor, a temperature sensor, an oxygen concentration sensor, an ion concentration sensor, an impedance sensor, a sensor adapted for cardiac output assessment, a blood filter, a septal occluder, a coil, a detachable coil for aneurysm treatment, a graft, and a deflector.

6. The self-expansible structure of claim 3, wherein said anchor further includes a carrier for mounting an intracorporeal device thereon.

7. The self-expansible structure of claim 1, wherein the nominal length is at least 25% greater than the nominal diameter.

8. The self-expansible structure of claim 1, wherein the nominal diagonal is at least 25% greater than the nominal diameter.

9. The self-expansible structure of claim 1, wherein said structure is formed of at least one closed wire construction, thus having no exposed tips.

10. The self-expansible structure of claim 1, wherein said structure is formed of at least two closed wire constructions, thus having no exposed tips.

11. The self-expansible structure of claim 1, wherein said structure defines a length axis, parallel to its length, and a point of midlength, on said length axis, and wherein said structure is symmetric with respect to a plane, orthogonal to said length axis, and crossing said length axis at said point of midlength.

12. The self-expansible structure of claim 1, wherein:
said structure is formed of at least one closed wire construction, thus having no exposed tips, and
said structure defines a length axis, parallel to its length, and a point of midlength, on said length axis, and wherein said structure is symmetric with respect to a plane, orthogonal to said length axis, and crossing said length axis at said point of midlength.

13. The self-expansible structure of claim 1, wherein said structure is formed of a shape memory alloy, having a martensite phase at temperatures below body temperature and an austenite phase at temperatures at and above body temperature, and wherein said structure is at the martensite phase, at the martensite temperatures, when constrained in said catheter, for insertion into the body.

14. The self-expansible structure of claim 13, wherein said structure is at the austenitic phase, at the austenitic temperatures, when implanted in the body cavity.

15. The self-expansible structure of claim 13, wherein said structure is at a stress-induced martensite phase, at the austenitic temperatures, when implanted in the body cavity.

16. The self-expansible structure of claim 1, wherein said structure is formed of a shape memory alloy, having an austenite phase and a stress induced martensite phase at and above room temperature, and wherein said structure is at the stress-induced martensite phase, both when constrained in said catheter, for insertion into the body, and when implanted in the body cavity.

17. The self-expansible structure of claim 1, wherein in the deployed state, a perimeter of said structure forms an arc that is at least as great as half the cavity perimeter.

18. The self-expansible structure of claim 1, wherein in the deployed state, a perimeter of said structure forms an arc that is at least as great as two thirds the cavity perimeter.

19. The self-expansible structure of claim 1, wherein said structure is formed of a wire of a diameter of between 0.03 and 1.0 mm.

20. The self-expansible structure of claim 1, wherein said structure is formed of a wire of a diameter of between 0.2 and 0.3 mm.

21. The self-expansible structure of claim 1, wherein the value of said transition diameter may be varied and controlled by the manner of constraining in said catheter, during insertion.

22. The self-expansible structure of claim 1, wherein said structure further includes at least one inner component for engaging with a retrieval instrument.

23. An implant system, comprising:
a self-expansible structure, for implantation in a body cavity, said structure comprising:
a generally tubular outline;
a nominal length;
a nominal diameter; and
a transition diameter, at which a constrained behavior of said structure changes so that:
at a constraining diameter smaller than said transition diameter, said structure conforms to the constraint by decreasing the structure's diameter below the nominal diameter and elongating beyond the nominal length, and
at a constraining diameter larger than said transition diameter but smaller than the nominal diameter, said structure conforms to the constraint by decreasing the structure's diameter below the nominal diameter, while substantially maintaining the nominal length,
wherein:
said structure is operable for insertion into a body cavity, via a catheter, having a catheter inner diameter smaller than said transition diameter, by decreasing the structure's diameter, while elongating, and
said structure is operable for implantation in the body cavity, having a cavity inner diameter greater than said transition diameter but smaller than the nominal diameter, by decreasing the structure's nominal diameter, while substantially maintaining the nominal length; and
an intracorporeal device, mounted on said self-expansible structure and adapted for performing a physiologically related task at the body cavity.

24. The implant system of claim 23, wherein said intracorporeal device is selected from the group consisting of a pressure sensor, a flow rate sensor, a temperature sensor, an oxygen concentration sensor, an ion concentration sensor, an impedance sensor, a sensor adapted for cardiac output assessment, a blood filter, a septal occluder, a coil, a detachable coil for aneurysm treatment, a graft, and a deflector.

25. The implant system of claim 23, wherein said intracorporeal device comprises at least two intracorporeal devices.

26. The implant system of claim 23, wherein said intracorporeal device comprises a power source.

27. The implant system of claim 23, wherein said intracorporeal device comprises an extracorporeally energizeable power source.

28. The implant system of claim 23, wherein said intracorporeal device is capable of telemetric communication with an extracorporeal device.

29. The implant system of claim 23, wherein the nominal length is at least 25% greater than the nominal diameter.

30. The self-expansible structure of claim 23, wherein the nominal diagonal is at least 25% greater than the nominal diameter.

31. The implant system of claim 23, formed of at least one closed wire construction, thus having no exposed tips.

32. The implant system of claim 23, formed of at least two closed wire constructions, thus having no exposed tips.

33. The implant system of claim 23, wherein said structure defines a length axis, parallel to its length, and a point of midlength, on said length axis, and wherein said structure is symmetric with respect to a plane, orthogonal to said length axis, and crossing said length axis at said point of midlength.

34. The implant system of claim 23, wherein:
said structure is formed of at least one closed wire construction, thus having no exposed tips, and
said structure defines a length axis, parallel to its length, and a point of midlength, on said length axis, and wherein said structure is symmetric with respect to a plane, orthogonal to said length axis, and crossing said length axis at said point of midlength.

35. The implant system of claim 23, wherein said structure is formed of a shape memory alloy, having a martensite phase at temperatures below body temperature and an austenite phase at temperatures at and above body temperature, and wherein said structure is at the martensite phase, at the martensite temperatures, when constrained in said catheter, for insertion into the body.

36. The implant system of claim 35, wherein said structure is at the austenitic phase, at the austenitic temperatures, when implanted in the body cavity.

37. The implant system of claim 35, wherein said structure is at a stress-induced martensite phase, at the austenitic temperatures, when implanted in the body cavity.

38. The implant system of claim 23, wherein said structure is formed of a shape memory alloy, having an austenite phase and a stress induced martensite phase at and above room temperature, and wherein said structure is at the stress-induced martensite phase, both when constrained in said catheter, for insertion into the body, and when implanted in the body cavity.

39. The implant system of claim 23, wherein in the deployed state, a perimeter of said structure forms an arc that is at least as great as half the cavity perimeter.

40. The implant system of claim 23, wherein in the deployed state, a perimeter of said structure forms an arc that is at least as great as two thirds the cavity perimeter.

41. The implant system of claim 23, wherein said structure is formed of a wire of a diameter of between 0.03 and 1.0 mm.

42. The implant system of claim 23, wherein said structure is formed of a wire of a diameter of between 0.2 and 0.3 mm.

43. The implant system of claim 23, wherein the value of said transition diameter may be varied and controlled by the manner of constraining in said catheter, during insertion.

44. The implant system of claim 23, wherein said structure further includes at least one inner component for engaging with a retrieval instrument.

45. A retrieval system, comprising:
a self-expansible structure, for implantation in a body cavity, said structure comprising:
a generally tubular outline;
a nominal length;
a nominal diameter; and
a transition diameter, at which a constrained behavior of said structure changes so that:
at a constraining diameter smaller than said transition diameter, said structure conforms to the constraint by decreasing the structure's diameter below the nominal diameter and elongating beyond the nominal length, and
at a constraining diameter larger than said transition diameter but smaller than the nominal diameter, said structure conforms to the constraint by decreasing the structure's diameter below the nominal diameter, while substantially maintaining the nominal length,
wherein:
said structure is operable for insertion into a body cavity, via a catheter, having a catheter inner diameter smaller than said transition diameter, by decreasing the structure's diameter, while elongating, and
said structure is operable for implantation in the body cavity, having a cavity inner diameter greater than said transition diameter but smaller than the nominal diameter, by decreasing the structure's nominal diameter, while substantially maintaining the nominal length,
wherein:
said structure includes at least one inner component for engaging with a retrieval instrument, and
said retrieval system includes a retrieval instrument, mounted on a retrieval catheter, adapted for percutaneous insertion into the body cavity, for engaging with said inner component and for retrieving said structure.

46. The retrieval system of claim 45, wherein said structure further includes an intracorporeal device, mounted thereon, adapted for performing a physiologically related task at the body cavity.

47. A method of implanting a self-expansible structure, comprising:
providing a self-expansible structure, for implantation in a body cavity, said structure comprising:
a generally tubular outline;
a nominal length;
a nominal diameter; and
a transition diameter, at which a constrained behavior of said structure changes so that:
at a constraining diameter smaller than said transition diameter, said structure conforms to the constraint by decreasing the structure's diameter below the nominal diameter and elongating beyond the nominal length, and
at a constraining diameter larger than said transition diameter but smaller than the nominal diameter, said structure conforms to the constraint by decreasing the structure's diameter below the nominal diameter, while substantially maintaining the nominal length;
constraining said structure within a catheter, having a catheter diameter smaller than said transition diameter, wherein said structure conforms to said catheter by decreasing the structure's diameter and elongating;
inserting said catheter and structure to a body cavity, having a cavity diameter larger than said transition diameter but smaller than the nominal diameter; and
deploying said structure in the body cavity, wherein said structure conforms to the cavity by decreasing the structure's diameter, while substantially maintaining the nominal length.

48. The method of claim 47, wherein said structure is a stent, designed to provide structural support to a blood vessel.

49. The method of claim 47, wherein said structure is an anchor, designed to support an intracorporeal device thereon.

50. The method of claim 49, wherein said anchor further includes an intracorporeal device, mounted thereon, adapted for performing a physiologically related task at the body cavity.

51. The method of claim 50, wherein said intracorporeal device is selected from the group consisting of a pressure sensor, a flow rate sensor, a temperature sensor, an oxygen concentration sensor, an ion concentration sensor, an impedance sensor, a sensor adapted for cardiac output assessment, a blood filter, a septal occluder, a coil, a detachable coil for aneurysm treatment, a graft, and a deflector.

52. The method of claim 49, wherein said anchor further includes a carrier for mounting an intracorporeal device thereon.

53. The method of claim 47, wherein the nominal length is at least 25% greater than the nominal diameter.

54. The method of claim 47, wherein the nominal diagonal is at least 25% greater than the nominal diameter.

55. The method of claim 47, wherein said structure is formed of at least one closed wire construction, thus having no exposed tips.

56. The method of claim 47, wherein said structure is formed of at least two closed wire constructions, thus having no exposed tips.

57. The method of claim 47, wherein said structure defines a length axis, parallel to its length, and a point of midlength, on said length axis, and wherein said structure is symmetric with respect to a plane, orthogonal to said length axis, and crossing said length axis at said point of midlength.

58. The method of claim 47, wherein:
said structure is formed of at least one closed wire construction, thus having no exposed tips, and
said structure defines a length axis, parallel to its length, and a point of midlength, on said length axis, and wherein said structure is symmetric with respect to a plane, orthogonal to said length axis, and crossing said length axis at said point of midlength.

59. The method of claim 47, wherein said structure is formed of a shape memory alloy, having a martensite phase at temperatures below body temperature and an austenite phase at temperatures at and above body temperature, and wherein said structure is at the martensite phase, at the martensite temperatures, when constrained in said catheter, for insertion into the body.

60. The method of claim 59, wherein said structure is at the austenitic phase, at the austenitic temperatures, when implanted in the body cavity.

61. The method of claim 59, wherein said structure is at a stress-induced martensite phase, at the austenitic temperatures, when implanted in the body cavity.

62. The method of claim 47, wherein said structure is formed of a shape memory alloy, having an austenite phase and a stress induced martensite phase at and above room temperature, and wherein said structure is at the stress-induced martensite phase, both when constrained in said catheter, for insertion into the body, and when implanted in the body cavity.

63. The method of claim 47, wherein in the deployed state, a perimeter of said structure forms an arc that is at least as great as half the cavity perimeter.

64. The method of claim 47, wherein in the deployed state, a perimeter of said structure forms an arc that is at least as great as two thirds the cavity perimeter.

65. The method of claim 47, wherein said structure is formed of a wire of a diameter of between 0.03 and 1.0 mm.

66. The method of claim 47, wherein said structure is formed of a wire of a diameter of between 0.2 and 0.3 mm.

67. The method of claim 47, wherein the value of said transition diameter may be varied and controlled by the manner of constraining in said catheter, during insertion.

68. The method of claim 47, wherein said method further includes retrieving said structure.

69. A self-expansible structure, for implantation in a body cavity, wherein said self-expansible structure is formed of at least one closed wire construction, thus having no exposed tips.

70. A self-expansible structure, for implantation in a body cavity, wherein said self-expansible structure is symmetric with respect to a plane, orthogonal to a length axis of said structure and crossing said length axis at a point of midlength of said structure.

71. A self-expansible structure, for implantation in a body cavity, wherein;
said self-expansible structure is formed of at least one closed wire construction, thus having no exposed tips; and
said self-expansible structure is symmetric with respect to a plane, orthogonal to a length axis of said structure and crossing said length axis at a point of midlength of said structure.

72. An implant system comprising:
a self-expansible structure, for implantation in a body cavity, wherein said self-expansible structure is formed of at least one closed wire construction, thus having no exposed tips; and
an intracorporeal device, mounted on said self-expansible structure and adapted for performing a physiologically related task at the body cavity.

73. An implant system comprising:
a self-expansible structure, for implantation in a body cavity, wherein said self-expansible structure is symmetric with respect to a plane, orthogonal to a length axis of said structure and crossing said length axis at a point of midlength of said structure; and
an intracorporeal device, mounted on said self-expansible structure and adapted for performing a physiologically related task at the body cavity.

74. An implant system comprising:
a self-expansible structure, for implantation in a body cavity, wherein;
said self-expansible structure is formed of at least one closed wire construction, thus having no exposed tips; and
said self-expansible structure is symmetric with respect to a plane, orthogonal to a length axis of said structure and crossing said length axis at a point of midlength of said structure; and
an intracorporeal device, mounted on said self-expansible structure and adapted for performing a physiologically related task at the body cavity.
